(19) United States
(12) Patent Application Publication
(10) Pub. No.: US 2019/0008804 A1
(43) Pub. Date: Jan. 10, 2019

(54) Trans-Clomiphene and Progesterone Receptor Antagonist Combination Therapy for Treating Hormone-Dependent Conditions

(71) Applicant: Repros Therapeutics Inc., The Woodlands, TX (US)

(72) Inventors: Joseph S. Podolski, The Woodlands, TX (US); Greg Fontenot, The Woodlands, TX (US)

(21) Appl. No.: 16/069,686

(22) PCT Filed: Jan. 11, 2017
(86) PCT No.: PCT/US17/12920

§ 371 Date: Jul. 12, 2018

Related U.S. Application Data
(60) Provisional application No. 62/277,782, filed on Jan. 12, 2016.

(51) Int. Cl. A61K 31/138; A61P 5/24; A61K 31/573; A61K 9/00

(52) U.S. Cl. CPC A61K 31/138; A61K 9/0053; A61K 31/573; A61P 5/24

(57) ABSTRACT

The present invention pertains to a combination for simultaneous, separate, or sequential use which comprises (a) trans-clomiphene or a salt or analogue thereof and (b) a progesterone receptor antagonist and to its use for the treatment of hormone-dependent conditions.

### FIELD OF THE INVENTION

This application claims the benefit of U.S. Provisional Application No. 62/277,782 filed Jan. 12, 2016, the contents of which are incorporated herein by reference.

In several embodiments, the invention relates to combination therapy with trans-clomiphene or an analogue thereof and a progesterone receptor antagonist for the treatment and/or prevention of a variety of hormone-dependent disorders.

### BACKGROUND OF THE INVENTION

Antiprogestins, compounds which inhibit the action of progesterone, have considerable potential for use in the pharmacological regulation of fertility and a variety of conditions and diseases such as breast cancer and endometriosis. The first reported antiprogestin, mifepristone (RU 486), is one of a number of 19-nortestsosterone derivatives with strong affinity for both the progesterone and glucocorticoid receptors and with antiprogestational and antiglucocorticoid activity. A variety of antiprogestins based on the 19-norprogesterone backbone have also been synthesized. Many of these compounds display mixed agonist/antagonist activity and are collectively known as selective progesterone receptor modulators (SPRMs). These compounds can be useful for treating a variety of progesterone-responsive conditions. Effective management of many of these conditions requires chronic administration of the SPRMs; however, long term oral administration of these compounds is associated with adverse effects limiting their usefulness.

Selective estrogen modulators (SERMs) are a class of compound that bind to estrogen receptors (ERs) thereby inducing specific conformational changes in the receptors. SERMs can exert different effects in different tissues resulting from tissue-specific recruitment of coactivators and corepressors. SERMs are therefore distinguished from the so-called “pure” estrogen receptor agonists/antagonists that uniformly activate or block estrogen effects independent of tissue type. SERMS, by virtue of their effect on the estrogen receptor, are useful for treating a variety of disorders having an estrogen component. Many of these disorders are chronic in nature and require long-term administration of the SERM. However, when administered over long periods of time, SPRMs cause adverse endometrial effects and break-through bleeding, limiting their long-term use.

There is a need in the art for improved treatment regimens for chronic estrogen and progesterone dependent conditions.

### SUMMARY OF THE INVENTION

In several embodiments, a combination therapy for use in the prevention and/or treatment of a hormone (i.e. estrogen and/or progesterone) dependent condition is provided comprising co-administering trans-clomiphene or an analogue or salt thereof and a progesterone receptor antagonist to a mammal in need of such treatment. In certain aspects, co-administration of trans-clomiphene or an analogue or salt thereof and a progesterone receptor antagonist to a mammal with a hormone dependent condition provides an enhanced and even synergistic effect compared to either treatment alone.

Trans-clomiphene or an analogue or salt thereof are administered simultaneously (separately or in the same formulation) or sequentially, in either order, to a patient in need thereof. In certain embodiments, the trans-clomiphene or analogue or salt thereof is administered prior to the progesterone antagonist. In other embodiments, the progesterone antagonist is administered prior to trans-clomiphene or an analogue or salt thereof.

Analogues of trans-clomiphene for use according to the invention include, without limitation, (E)-Clomiphene-NO, (E)-di-desethyl Clomiphene, (E)-desethyl Clomiphene, (E)-4-OH-Clomiphene and (E)-4-OH-desethyl Clomiphene. Preferred trans-clomiphene analogs for use according to the invention are (E)-4-OH-Clomiphene and (E)-4-OH-desethyl Clomiphene. Preferred trans-clomiphene salts include citrate salt and phosphate salt.

In several embodiments, the progesterone receptor antagonist is a selective progesterone receptor modulator (SPRM) and is preferably CDB-4124 (21-methoxy-17α-acetoxy-11β-(4 N, N-dimethylaminophenyl)-19-norpregna-4,9-diene-3,20-dione; telapristone). A preferred salt of CDB-4124 for use in the methods is the acetate salt (telapristone acetate). Another preferred salt of CDB-4124 is the phosphate salt.

In preferred embodiments, the present invention provides methods for treating or preventing a hormone dependent disorder comprising orally administering trans-clomiphene or a salt or analogue thereof and simultaneously or sequentially orally administering a progesterone receptor antagonist to a patient in need of such treatment.

In preferred embodiments, the combination therapy is administered to a female, preferably a human female, in order to treat and/or prevent a hormone dependent condition in the female. Hormone-dependent conditions that may be treated and/or prevented by combination therapy of the invention include, without limitation, endometriosis and pain associated therewith, adenomyosis, endometriomas of the ovary, dysmenorrhea, endocrine hormone-dependent tumors, uterine fibroids, endometrial hyperproliferation, menorrhagia, ovarian cancer, cervical cancer and breast cancer. Combination therapy according to the invention can also be administered for contraception.

Combination therapy according to the invention can be administered chronically. In several embodiments, the treatment period is at least 1, 2, 3, 4, 5, 6, 7, 8, 9, 10, 11, or 12 months, 2, 3, 4, or 5 years or any range there between.

Also provided is a pharmaceutical composition comprising a therapeutically effective amount of trans-clomiphene or an analogue or pharmaceutically acceptable salt thereof and a progesterone receptor antagonist (e.g. CDB-4124).

In a further aspect, a kit for use in treating a hormone dependent condition in a subject is provided including trans-clomiphene or an analogue or salt thereof and a progesterone antagonist (e.g. CDB-4124). The kit may further comprise instructions for using the combination for treating a hormone-dependent condition such as endometriosis or uterine fibroids.

### BRIEF DESCRIPTION OF THE DRAWINGS

FIGS. 1A-1B. FIGS. 1A and 1B illustrate body composition of female baboons at baseline (pre-treatment) and after six months of daily oral administration of 12 mg Proellex (CDB-4124) and 25 mg enclomiphene.

FIGS. 2A-2D illustrate fat distribution of female baboons at baseline (pre-treatment) and after six months of daily oral administration of Placebo (FIG. 2A) Proellex alone (12 mg) (FIG. 2B), Androxal alone (25 mg) (FIG. 2C), or combined administration of Proellex and Androxal (12 mg, 25 mg) (FIG. 2D).

FIGS. 3A-3D illustrate bone mineral density (BMD) in female baboons at baseline (pre-treatment) and after six months of daily oral administration of Placebo (FIG. 2A) Proellex alone (12 mg) (FIG. 2B), Androxal alone (25 mg) (FIG. 2C), or combined administration of Proellex and Androxal (12 mg, 25 mg) (FIG. 2D)

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

It is to be understood that any ranges, ratios and ranges of ratios that can be formed by any of the numbers or data present herein represent further embodiments of the present invention. This includes ranges that can be formed that do or do not include a finite upper and/or lower boundary. Accordingly, the skilled person will appreciate that many such ratios, ranges and ranges of ratios can be unambiguously derived form the data and numbers presented herein and all represent embodiments of the invention.

Before the present compounds, compositions and methods are disclosed and described, it is to be understood that the terminology used herein is for the purpose of describing particular embodiments only and is not intended to be limiting. It must be noted that, as used in the present specification and the appended claims, the singular forms “a,” “an” and “the” include plural referents unless the context clearly dictates otherwise.

Definitions

It is to be understood that “combination therapy” envisages the simultaneous, sequential or separate administration of the components of the combination. In one aspect of the invention, “combination therapy” envisages simultaneous administration of trans-clomiphene or an analogue or salt thereof and a progesterone antagonist. In a further aspect of the invention, “combination therapy” envisages sequential administration of trans-clomiphene or an analogue or salt thereof and a progesterone antagonist. In another aspect of the invention, “combination therapy” envisages separate administration of trans-clomiphene or an analogue or salt thereof and a progesterone antagonist. Where the administration of trans-clomiphene or an analogue or salt thereof and a progesterone antagonist is sequential or separate, trans-clomiphene or an analogue or salt thereof and a progesterone antagonist are administered within time intervals that allow that the therapeutic agents show a cooperative e.g., synergistic, effect. In preferred embodiments, trans-clomiphene or an analogue or salt thereof and a progesterone antagonist are administered within 1, 2, 3, 6, 12, 24, 48, 72 hours, or within 4, 5, 6 or 7 days or within 8, 9, 10, 11, 12, 13, 14, 15, 16, 17, 18, 19, 20, 21, 22, 23, 24, 25, 26, 27, 28, 29, 30 or 31 days of each other.

The term “oral” administration means that the active agent is in a formulation designed to be ingested, i.e. designed to be delivered to the gastrointestinal system for absorption.

The term “effective dosage” means an amount of the composition's active component sufficient to treat a particular condition.

The term “pharmaceutically acceptable salt” refers to a salt prepared from a pharmaceutically acceptable non-toxic inorganic or organic acid. Inorganic acids include, but are not limited to, hydrochloric, hydrobromic, hydroiodic, nitric, sulfuric, and phosphoric. Organic acids include, but are not limited to, aliphatic, aromatic, carboxylic, and sulfonic organic acids including, but not limited to, formic, acetic, propionic, succinic, benzoic camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic, stearic, sulfanilic, alginic, and galacturonic acid. Preferred salts are the citrate salt and phosphate salts in the case of trans-clomiphene or analogues thereof. Preferred salts are the acetate and phosphate salts in the case of CDB-4124.

The term “selective progesterone receptor modulators” or “SPRMs” means compounds that affect functions of progesterone receptor in a tissue-specific manner. The compounds act as progesterone receptor antagonists in some tissues (for example, in breast tissue) and as progesterone receptor agonists in other tissues (for example, in the uterus).

The term “treat” or “treatment” as used herein refers to any treatment of any hormone-dependent disorder or disease, and includes, but is not limited to, inhibiting the disorder or disease arresting the development of the disorder or disease; relieving the disorder or disease, for example, causing regression of the disorder or disease; or relieving the condition caused by the disease or disorder, relieving the symptoms of the disease or disorder.

The term “prevent” or “prevention,” in relation to a hormone-dependent disorder or disease, means preventing the onset of disorder or disease development if none had occurred, or preventing further disorder or disease development if the disorder or disease was already present.

The present invention provides a combination therapy for treating or preventing hormone-dependent conditions including without limitation, endometriosis and pain associated therewith, dysfunctional uterine bleeding, adenomyosis, endometriomas of the ovary, dysmenorrhea, menorrhagia, endocrine hormone-dependent tumors, uterine fibroids, endometrial hyperproliferation, ovarian cancer, cervical cancer and breast cancer. The present invention also provides a combination for use in a method of contraception.

In several embodiments, the present methods utilize one or more progesterone receptor antagonists, defined herein as compounds that bind to a progesterone receptor and inhibit the effect of progesterone. Progesterone receptor antagonists include so-called “pure” antiprogestins such as mifepristone, as well as selective progesterone receptor modulators (SPRMs) such as ulipristal, asoprisnil and CDB-4124 which may act as progesterone receptor agonists in certain tissues and progesterone receptor antagonists in others. The methods are particularly useful for long-term (chronic) administration of selective progesterone receptors.

Non-limiting examples of progesterone receptor antagonists include the steroid compounds disclosed in U.S. Pat. Nos. 6,861,415 and 6,900,193, the contents of which are incorporated herein by reference. In a preferred embodiment, the progesterone receptor antagonist is an SPRM selected from CDB-4124 (21-methoxy-17α-acetoxy-11β-(4 N, N-dimethylaminophenyl)-19-norpregna-4,9-diene-3,20-dione; telapristone), CDB-2914 (17α-acetoxy-11β-(4-N,N-dimethylaminophenyl)-19-norpregna-4,9-dien-3,20-dione; ulipristal), CDB-4453 (21-methoxy-17α-acetoxy-11β-(4-N-methylaminophenyl)-19-norpregna-4,9-diene-3,20-dione) and asoprisnil (benzaldehyde, 4-[(11β,17β)-17-methoxy-17-(methoxymethyl)-3-oxoestra-4,9-dien-11-yl]-1-(E)-oxim; J867).

Other preferred progesterone receptor antagonists for practicing the methods of the invention include, without limitation, Mifepristone (RU-486; 11β-[4 N,N-dimethylaminophenyl]-17β-hydroxy-17-(1-propynyl)-estra-4,9-dien-3-one), Lilopristone (11β-(4 N,N-dimethylaminophenyl)-17β-hydroxy-17-((Z)-3-hydroxypropenyl)estra-4,9-dien-3-one), Onapristone (11β-(4 N,N-dimethylaminophenyl)-17α-hydroxy-17-(3-hydroxypropyl)-13α-estra-4,9-dien-3-one), J912 (4-[17β-Hydroxy-17α-(methoxymethyl)-3-oxoestra-4,9-dien-11β-yl]benzaldehyd-(1E)-oxim).

Other antiprogestins include compounds described in U.S. Pat. Nos. 4,386,085, 4,447,424, 4,536,401, 4,519,946, 4,609,651, 4,634,695, 4,780,461, 4,814,327, 4,829,060, 4,871,724, 4,921,845, 4,921,845, 5,095,129, 5,446,178, 5,478,956, 5,232,915 5,089,488, 5,093,507, 5,244,886, 5,292,878, 5,439,913, 5,446,036, 5,576,310; 5,684,151, 5,688,808, 5,693,646, 5,693,647, 5,696,127, 5,696,130, 5,696,133 5,739,125, 5,407,928, 5,273,971, 5,728,689, 5,753,655, 5,843,933, 5,843,931, 6,509,334, 6,566,358, 6,713,478, 6,391,907, 6,417,214, 6,380,235, 6,339,098, 6,306,851, 6,441,019, 6,369,056, and 6,358,948, the contents of each of which are incorporated herein by reference.

Yet other progesterone receptor antagonists useful in practicing the methods of the invention, include without limitation JNJ-1250132, (6α,11β,17β)-11-(4-dimethylaminophenyl)-6-methyl-4′,5′-dihydrospiro[estra-4,9-diene-17,2′(3′H)-furan]-3-one (ORG-31710); (11β,17α)-11-(4-acetylphenyl)-17,23-epoxy-19,24-dinorchola-4,9,20-trien-3-one (ORG-33628); (7β,11β,17β)-11-(4-dimethylaminophenyl-7-methyl]-4′,5′-dihydrospiro[estra-4,9-diene-17,2′(3′H)-furan]-3-one (ORG-31806); ZK-112993; ORG-31376; ORG-33245; ORG-31167; ORG-31343; RU-2992; RU-1479; RU-25056; RU-49295; RU-46556; RU-26819; LG1127; LG120753; LG120830; LG1447; LG121046; CGP-19984A; RTI-3021-012; RTI-3021-022; RTI-3021-020; RWJ-25333; ZK-136796; ZK-114043; ZK-230211; ZK-136798; ZK-98229; ZK-98734; ZK-137316; 4-[17β-Methoxy-17α-(methoxymethyl)-3-oxoestra-4,9-dien-11β-yl]benzaldehyde-1-(E)-oxime; 4-[17β-Methoxy-17α-(methoxymethyl)-3-oxoestra-4,9-dien-11β-yl]benzaldehyde-1-(E)-[O-(ethylamino)carbonyl]oxime; 4-[17β-Methoxy-17α-(methoxymethyl)-3-oxoestra-4,9-dien-11β-yl]benzaldehyde-1-(E)[O-(ethylthio)carbonyl]oxime; (Z)-6′-(4-cyanophenyl)-9,11α-dihydro-17β-hydroxy-17α-[4-(1-oxo-3-methylbutoxy)-1-butenyl]4′H-naphtho[3′,2′,1′;10,9,11]estr-4-en-3-one; 11β-(4-acetylphenyl)-17β-hydroxy-17α-(1,1,2,2,2-pentafluoroethyl)estra-4,9-dien-3-one; 11beta-(4-Acetylphenyl)-19,24-dinor-17,23-epoxy-17alpha-chola-4,9,20-trien-3-one; (Z)-11beta,19-[4-(3-Pyridinyl)-o-phenylene]-17beta-hydroxy-17α-[3-hydroxy-1-propenyl]-4-androsten-3-one; 11beta-[4-(1-methylethenyl)phenyl]-17α-hydroxy-17beta-(3-hydroxypropyl)-13α-estra-4,9-dien-3-one; 4′,5′-Dihydro-11beta-[4-(dimethylamino)phenyl]-6beta-methylspiro[estra-4,9-dien-17beta,2′(3′H)-furan]-3 -one.

Also useful with the methods of the invention are salts of progesterone receptor antagonists. Depending on the process conditions the salt compound obtained may be either in neutral or salt form. Salt forms include hydrates and other solvates and also crystalline polymorphs. Both the free base and the salts of these end products may be used in accordance with the invention.

Acid addition salts may in a manner known per se be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs may be used in accordance with the invention. A preferred salt is the acetate salt. Another preferred salt is the phosphate salt.

Base addition salts may also be used in accordance with the invention and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like.

Administration of the progesterone receptor antagonist may be accomplished by daily administration, periodic administration (i.e., administration at uniform intervals less frequent than daily such as every other day, weekly, bi-weekly or monthly) or intermittent administration by which it is meant that the progesterone antagonist is administered continuously, either daily or periodically, for an administration period then administration of the progesterone antagonist is discontinued for a period of time greater than the dosing interval during the previous administration period but less than the administration period, then the progesterone antagonist is administered daily or periodically for an administration period, then administration is discontinued and so on. For the treatment of endometriosis and pain associated therewith, adenomyosis, endometriomas of the ovary, dysmenorrhea, uterine fibroids, endometrial hyperproliferation, ovarian cancer, and cervical cancer, systemic administration is preferably accomplished by administering the progesterone antagonist daily or every other day, preferably orally.

Therapeutically effective doses of the progesterone receptor antagonist may be between 3 mg and 100 mg per day, between 3 mg and 75 mg per day, between 3 mg and 50 mg per day, between 3 mg and 25 mg per day, between 3 mg and 12 mg per day, between 5 mg and 100 mg per day, between 5 mg and 75 mg per day, between 5 mg and 50 mg per day, between 5 mg and 25 mg per day, between 5 mg and 12 mg per day, or any range there between. In preferred embodiments, the progesterone receptor antagonist is administered at a dosage less than 50 mg/day, less than 40 mg/day, less than 30 mg/day less than 20 mg/day, less than 10 mg/day, or less than 5 mg/day. Preferred embodiments comprise administering an SPRM (e.g. CDB-4124) at a dosage of between 3 and 50 mg per day, preferably between 3 and 25 mg per day.

Therapeutically effective doses of trans-clomiphene (or salt or analogue thereof) are from 5 mg to 100 mg, 5 mg to 50 mg, 5 mg to 25 mg, 5 mg to 12 mg, 10 mg to 100 mg, 10 mg to 50 mg, 10 mg to 25 mg, 12.5, 25 mg, or 50 mg, or any range there between, preferably administered daily or every other day.

Co-administration (sequential or simultaneous) of trans-clomiphene (or salt or analogue thereof) and a progesterone receptor antagonist may be accomplished by any appropriate route, including but not limited to injection (preferably intramuscular), oral, transdermal (e.g. patches), topical (e.g. gels and creams) and transmucosal (e.g. vaginal) administration. Trans-clomiphene (or salt or analogue thereof) and progesterone receptor antagonist may be administered by the same or different route. For example, trans-clomiphene (or salt or analogue thereof) may be administered orally and progesterone receptor antagonist may be administered vaginally. In a preferred embodiment, trans-clomiphene (or salt or analogue thereof) and a progesterone receptor antagonist are both administered orally.

Therapeutically effective doses of the antiprogestin when administered locally (e.g. to the vaginal mucosa to treat uterine fibroids) may between 3 mg/day and 50 mg/day, between 3 mg/day and 40 mg/day, between 3 mg/day and 30 mg/day, between 3 mg/day and 20 mg/day, or between 3 mg/day and 12 mg/day or between 5 mg/day and 12.5 mg/day.

In one embodiment, a combination therapy as described herein is administered to a female to suppress endometrial proliferation. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

In a related embodiment, a combination therapy as described herein is administered to a female to treat endometriosis. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

In a related embodiment of the invention, a combination therapy as described herein is administered to a female patient in need thereof according to the present methods in order to treat dysmenorrhea. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

In a related embodiment of the invention, a combination therapy as described herein is administered to a female patient in need thereof according to the present methods in order to treat uterine fibroids. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

In a related embodiment of the invention, a combination therapy as described herein is administered to a female patient in need thereof according to the present methods in order to treat dysfunctional uterine bleeding. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

In another embodiment, a combination therapy as described herein is administered to a female patient desiring contraception according to the present methods. In a preferred embodiment, the method does not comprise also administering the female an estrogen. The combination therapy according to the present invention effectively prevents cycling while minimizing or even avoiding adverse endometrial effects accompanying chronic administration of SPRMs and the loss in bone mineral density accompanying traditional contraceptive methods wherein estrogen is not co-administered. In a preferred embodiment, the progesterone receptor antagonist is a selective progesterone receptor modulator (SPRM) at a dose of from about 5 mg to about 25 mg or from about 3 mg to 12.5 mg per day. In a related aspect, the SPRM is CDB-4124, CDB-2914 or asoprisnil.

For local administration, the progesterone antagonist may be prepared in any formulation suitable for local administration. For example, the compound may be formulated, without limitation, as an intravaginal preparation such as a doughnut-shaped hormone-releasing vaginal ring; a vaginal suppository; a vaginal pill; an intra-uterine preparation such as an intrauterine device (IUD) or matrix preparation; an implantable drug delivery device; a topical gel; a cream, an ointment, a trans-dermal patch or in a bioadhesive carrier such as those described in U.S. Pat. No. 4,615,697, which is incorporated herein by reference. The bioadhesive carrier may be in gel, cream, tablet, pill, capsule (e.g. pullulan capsule), suppository, or film form or any other pharmaceutically acceptable form that will adhere to the vaginal mucosa. Preferably the formulation comprises a unit dose of the progesterone antagonist of between 3 mg and 25 mg, or any range there between, such as 3 mg, 5 mg, 8 mg, 12 mg, 15 mg, 20 mg or 25 mg and one or more pharmaceutically acceptable carriers.

For systemic administration, trans-clomiphene (or a salt or analogue thereof) or a progesterone receptor antagonist may be prepared in the form of a dose unit or dose units suitable for systemic administration. For example, the compound may be formulated in a solid dosage unit suitable for oral administration such as a tablet (e.g. standard hard tablets, suspension tablets, rapid dispersion tablets, chewable tablets, effervescent tablets, bilayer tablets, etc.), caplet, capsule (e.g., a soft or a hard gelatin capsule), powder (e.g. a packaged powder, a dispensable powder or an effervescent powder), lozenge, sachet, cachet, troche, pellet granules, microgranules, encapsulated microgranules, or any other solid dosage form. Alternatively, the compound may be formulated in suitable liquid dosage forms such as solutions, aqueous suspensions, elixirs, syrups, etc. Preferably the formulation comprises a unit dose of the progesterone receptor antagonist of between 3 mg and 25 mg, or any range there between, such as 3 mg, 5 mg, 8 mg, 12 mg, 15 mg, 20 mg or 25 mg and one or more pharmaceutically acceptable carriers and/or comprises a unit dose of trans-clomiphene or a salt or analogue thereof of between 5 mg and 100 mg or any range there between, such as 5 mg, 10 mg, 12 mg 12.5 mg, 15 mg, 20 mg, 25 mg, 30 mg, 40 mg, or 50 mg and one or more pharmaceutically acceptable carriers.

Compositions of the invention can, if desired, include one or more pharmaceutically acceptable excipients. The tern “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition. Excipients include, by way of illustration and not limitation, diluents, disintegrants, binding agents, adhesives (e.g. bioadhesives), wetting agents, lubricants, glidants, surface modifying agents or surfactants, fragrances, suspending agents, emulsifying agents, nonaqueous vehicles, preservatives, antioxidants, adhesives, agents to adjust pH and osmolarity (e.g. buffering agents), preservatives, thickening agents, sweetening agents, flavoring agents, taste masking agents, colorants or dyes, penetration enhancers and substances added to improve appearance of the composition.

A therapeutically effective amount of the composition required for use in therapy varies with the length of time that activity is desired, and the age and the condition of the patient to be treated, among other factors, and is ultimately determined by the attendant physician. In general, however, doses employed for human treatment typically are in the range of about 0.001 mg/kg to about 500 mg/kg per day, for example about 1 μg/kg to about 1 mg/kg per day or about 1 μg/kg to about 100 μg/kg per day. For most large mammals, the total daily dosage is from about 1 to 100 mg, preferably from about 2 to 80 mg, more preferably from about 3 to about 25 mg. The dosage regimen may be adjusted to provide the optimal therapeutic response. The desired dose may be conveniently administered in a single dose, or as multiple doses administered at appropriate intervals, for example as two, three, four or more subdoses per day.

Patients undergoing treatments with the compositions of the instant invention should be monitored routinely for their serum estrogen and glucocorticoid levels.

The following non-limiting examples are provided to aid in understanding the teachings of the instant invention.

### EXAMPLE 1

### Estrogen Receptor Binding Affinities and Co-Activator Studies

Studies were conducted to evaluate the binding affinity of trans-clomiphene (enclomiphene) and several analogues thereof to ERα and ERβ using in vitro competitive radioligand binding assays with [³H]estradiol (a natural high affinity ER ligand) and ERα or ERβ ligand binding domains expressed in insect Sf9 cells.

IC₅₀ values were determined by non-linear least squares regression analysis. Inhibition constants (Kᵢ) were calculated using the equation of Cheng and Prusoff (Cheng et al., Biochem. Pharmacol., 22:3099-3108 (1973)) using the observed IC₅₀ of the tested compound, the concentration of radioligand employed in the assay and the historical values for the K_{D} of the ligand. The Hill coefficient (n_{H}) defines the slope of the competitive binding curve and was determined using MathIQ™ (ID Business Solutions Ltd., UK).

The data are presented below and reflect the results of three separate radioligand binding studies:

| | Receptor | | Receptor | | Receptor | |
|---|---|---|---|---|---|---|
| | Binding | | Binding | | Binding | |
| | ERα | ERβ | ERα | ERβ | ERα | ERβ |
| | IC₅₀ | IC₅₀ | IC₅₀ | IC₅₀ | IC₅₀ | IC₅₀ |
| | nM | nM | nM | nM | nM | nM |
| Enclomiphene | 1.65 | — | — | — | — | — |
| 4-OH- | 0.34 | 0.57 | 0.68 | 0.41 | — | — |
| enclomiphene | | | | | | |
| Di-de-ethyl- | 4.88 | — | — | — | — | — |
| enclomiphene | | | | | | |
| Enclomiphene- | 9.61 | — | — | — | — | — |
| NO | | | | | | |
| 4-OH-N- | — | — | — | — | 0.20 | 0.45 |
| Desethyl | | | | | | |
| enclomiphene | | | | | | |
| N-Desethyl | — | — | — | — | 0.33 | 1.13 |
| enclomiphene | | | | | | |

Additional data obtained for each compound in the studies is presented below in rank order of potency:

| Compound | Species | Conc. | % Inh. | IC₅₀ | Kᵢ | n_{H} |
|---|---|---|---|---|---|---|
| ERα | | | | | | |
| 4-OH | hum | 0.3 nM | 51 | 0.34 nM | 0.096 | 1.06 |
| enclomiphene | | | | | | |
| Enclomiphene | hum | 3.6 nM | 69 | 1.65 nM | 0.47 nM | 0.95 |
| di-de-ethyl- | hum | 9 nM | 64 | 4.88 nM | 1.39 nM | 0.91 |
| enclomiphene | | | | | | |
| E-clom-NO | hum | 0.012 μM | 56 | 9.61 nM | 2.75 nM | 1.15 |
| Mono-de- | hum | 0.012 μM | 51 | 0.011 μM | 3.26 nM | 1.10 |
| ethyl-4OH- | | | | | | |
| enclomiphene | | | | | | |
| ERβ | | | | | | |
| 4-OH | hum | 0.9 nM | 67 | 0.57 nM | 0.12 nM | 1.56 |
| enclomiphene | | | | | | |

ERα and ERβ co-activator responses were also assessed for these compounds. The data is presented below and reflects the results of two separate studies:

| | ERα Coactivator | | | ERβ Coactivator | | | ERα Coactivator | | | ERβ Coactivator | | |
|---|---|---|---|---|---|---|---|---|---|---|---|---|
| | Response | | | Response | | | Response | | | Response | | |
| Compound | | | IC₅₀/EC₅₀ | | | IC₅₀/EC₅₀ | | | IC₅₀/EC₅₀ | | | IC₅₀/EC₅₀ |
| Name | Ag | Ant | nM | Ag | Ant | nM | Ag | Ant | nM | Ag | Ant | nM |
| Enclomiphene | ND | 73% | 16 | ND | 65% | 59 | — | — | — | — | — | — |
| 4-OH | ND | 59% | 2.21 | ND | 60% | 0.53 | — | — | — | — | — | — |
| Enclomiphene | | | | | | | | | | | | |
| Di-de-ethyl- | ND | 96% | 140 | ND | 87% | 230 | — | — | — | — | — | — |
| En- | | | | | | | | | | | | |
| Clomiphene | | | | | | | | | | | | |
| Enclomiphene- | ND | 54% | 54 | ND | 95% | 130 | — | — | — | — | — | — |
| NO | | | | | | | | | | | | |
| 4-OH N- | — | — | — | — | — | — | ND | 63% | 0.77 | ND | 104% | 1.3 |
| Desethyl | | | | | | | | | | | | |
| Enclomiphene | | | | | | | | | | | | |
| N-Desethyl | — | — | — | — | — | — | ND | 76% | 22 | ND | 74% | 40 |
| enclomiphene | | | | | | | | | | | | |

ND = Assay not done;
Ag = agonist;
Ant = antagonist

Methods

Estrogen ERα
Source: Human recombinant insect Sf9 cells
Vehicle: 1.00% DMSO
Incubation Time/Temp: 2 hours@25 C
Incubation Buffer: 10 mM Tris-HCl, pH 7.4, 0.1% BSA, 10% Glycerol, 1 mM DTT
Kd: 0.20 nM (historic value)
Ligand: 0.50 nM [³H] Estradiol
Non-Specific Ligand: 1.0 μM Diethylstilbestrol
Specific Binding: 85%
Quantitation Method: Radioligand Binding
Significance Criteria: >50% of max stimulation or inhibition
Bmax: 1400 pmole/mg Protein

Estrogen ERβ
Source: Human recombinant insect Sf9 cells

Estrogen ERβ
Source: Human recombinant insect Sf9 cells
Vehicle: 1.00% DMSO
Incubation Time/Temp: 2 hours@25 C
Incubation Buffer: 10 mM Tris-HCl, pH 7.4, 0.1% BSA, 10% Glycerol, 1 mM DTT
Kd: 0.13 nM (historic value)
Ligand: 0.50 nM [³H] Estradiol
Non-Specific Ligand: 1.0 μM Diethylstilbestrol
Specific Binding: 90%
Quantitation Method: Radioligand Binding
Significance Criteria: ≥50% of max stimulation or inhibition
Bmax: 3000 pmole/mg Protein

### Reference Compounds

| | | | | | Concur- |
|---|---|---|---|---|---|
| Assay | | Historical | | | rent |
| Name | Reference Cmpd | IC₅₀* | Kᵢ | n_{H} | IC₅₀ |
| Estrogen | Diethylstilbestrol | 0.77 nM | 0.22 nM | 1.0 | 10.9 nM |
| ERα | | | | | |
| Estrogen | Diethylstilbestrol | 0.61 nM | 0.13 nM | 1.20 | 0.64 nM |
| ERβ | | | | | |

The 4-OH derivative of enclomiphene and subsequent mono dethylated derivative of the 4-OH metabolite are the two most active molecules exhibiting binding to the ERα receptor approaching 10× that of the parent molecule.

Based on the binding and co-activator studies, the order of activity at the ERα receptor was determined to be: 4-OH-N-des-ethyl-enclomiphene>4-OH-enclomiphene>des-ethyl-enclomiphene>enclomiphene>>di-des-ethyl-enclomiphene>NO-enclomiphene. Most of the compounds are better ligands for ERα than ERβ with the exception of 4-OH-enclomiphene, which was a better ligand for ERβ. These compounds are all ERα/β antagonists. The data indicate that enclomiphene and its metabolites may tend to act biologically as true antiestrogens rather than SPRMs as previously thought.

### EXAMPLE 2

### Uterotrophic Response of Clomiphene Isomers in Mice

The Uterotrophic response to Enclomiphene citrate compared to Zuclomiphene citrate and Estradiol benzoate in ovariectomized (OVX) mice is investigated. The study is conducted to determine estrogenic effects of the clomiphene isomers.

Female C57BL/6J mice weighing 16-18 grams are divided into six Groups (I-VI; n=10/group). Mice are ovariectomized 14 days prior to compound administration (30 days).

Group I: Sham surgery (ovaries intact)-Sesame seed oil injected

Group 2: Ovariectomized-Sesame seed oil injected

Group 3: Ovariectomized-Estradiol benzoate (0.81 μg)

Group 4: Ovariectomized-Enclomiphene citrate (20 MPK)

Group 5: Ovariectomized-Zuclomiphene citrate (20 MPK)

Group 6: Ovariectomized-Tamoxifen (20 MPK)

Tissues are analyzed: Uterus (collected from uterotubal junction to cervix) and Ovary (without fallopian tubes). Wet tissue weight (with fluid expressed from uteri) and Histology (H&E) are assessed.

Estradiol benzoate is dissolved in sesame oil and injected subcutaneously twice daily for three days then once per day for the remaining time. Clomiphene isomers and tamoxifen are administered orally daily at 40 MPK for 30 days.

### EXAMPLE 3

### Bone Effects of Androxal and Proellex

Twelve female pigs were assigned to one of four groups (n=3): (1) placebo (2) Proellex (CDB-4124) (3) Androxal (trans-clomiphene) and (4) Androxal+Proellex. Active agents or placebo were administered after heat (estrus) in the luteal phase. Group 1 received orally administered placebo capsules; Group 2 received 12 mg of Proellex administered orally; Group 3 received 25 mg Androxal capsules administered orally; Group 4 were administered a combination of 12 mg Proellex (CDB-4124) and 25 mg Androxal (trans-clomiphene). All treatment groups received daily oral administration of the appropriate capsule(s) for 180 days. Daily general health observations and weekly body weight measurements were conducted for all groups. Following the 6 month administration period, calcium (parts per million (ppm) dry weight (dw)) was determined in rib and femur bone samples from pigs in each treatment group:

| Treatment (n = 3) | | Ca (ppm dw) |
|---|---|---|
| Placebo | Mean | 136333.33 |
| | SD | 8504.90 |
| Proellex 12 mg | Mean | 129166.67 |
| | SD | 4310.84 |
| | p< | 0.26 |
| Androxal 25 mg | Mean | 139300.00 |
| | SD | 14891.94 |
| | p< | 0.78 |
| Proellex 12 mg + | Mean | 124166.67 |
| Androxal 25 mg | | |
| | SD | 10408.33 |
| | p< | 0.19 |

Enclomiphene and CDB-4124, alone or in combination, did not affect the calcium content of bone. This is surprising because tamoxifen and other antiestrogens as well as GnRH antagonists result in significant bone loss which has limited their chronic use or even rendered them unsuitable as therapeutic agents altogether.

### EXAMPLE 4

### Study—Co-Administration of Proellex and Androxal in Cycling Female Baboons

Twelve normal, cycling female baboons of prime reproductive age were assigned to one of four groups (n=3): (1) placebo (2) Prellex (CDB-4124) (3) Androxal (trans-clomiphene) and (4) Androxal+Proellex. Group 1 received orally administered placebo capsules; Group 2 received 12 mg of Proellex administered orally; Group 3 received 25 mg Androxal capsules administered orally; Group 4 were administered a combination of 12 mg Proellex (CDB-4124) and 25 mg Androxal (trans-clomiphene). All treatment groups received daily oral administration of the appropriate capsule(s) for 180 days. Daily oral dosing was started in the luteal phase.

Pre-dose, and following the 6 month administration period, the baboons underwent ovarian ultrasound (for detection of ovarian cysts), DEXA, endometrial biopsy, and blood collection for analysis (pharmacokinetic analysis, blood chemistries, blood drug levels and hormonal analysis including LH, FSH, testosterone, estrogen, prolactin, and progesterone) at Months 1, 2, 3, 4, 5 and 6. Daily observations of estrus (heat) were conducted for all groups.

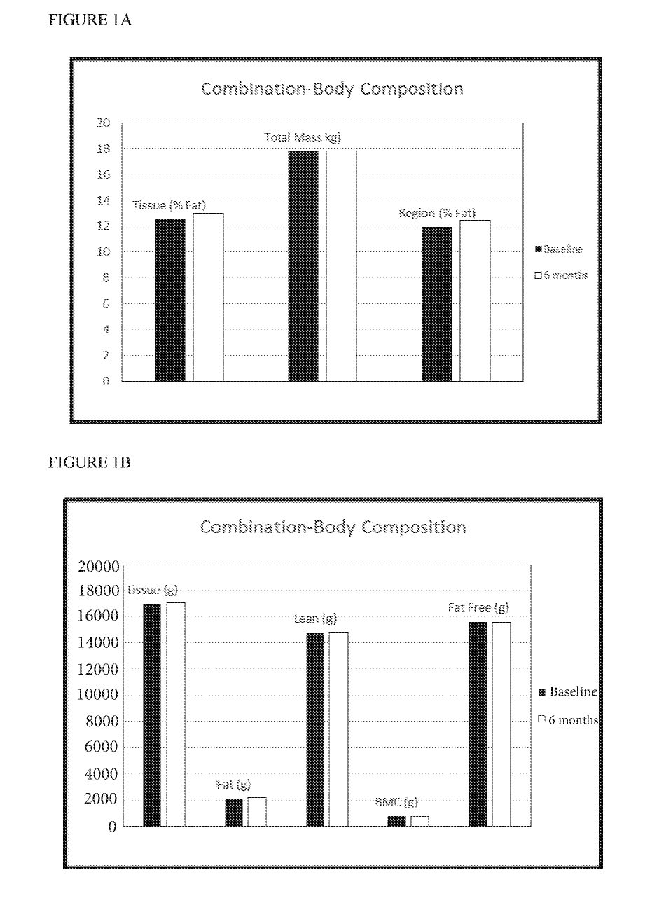

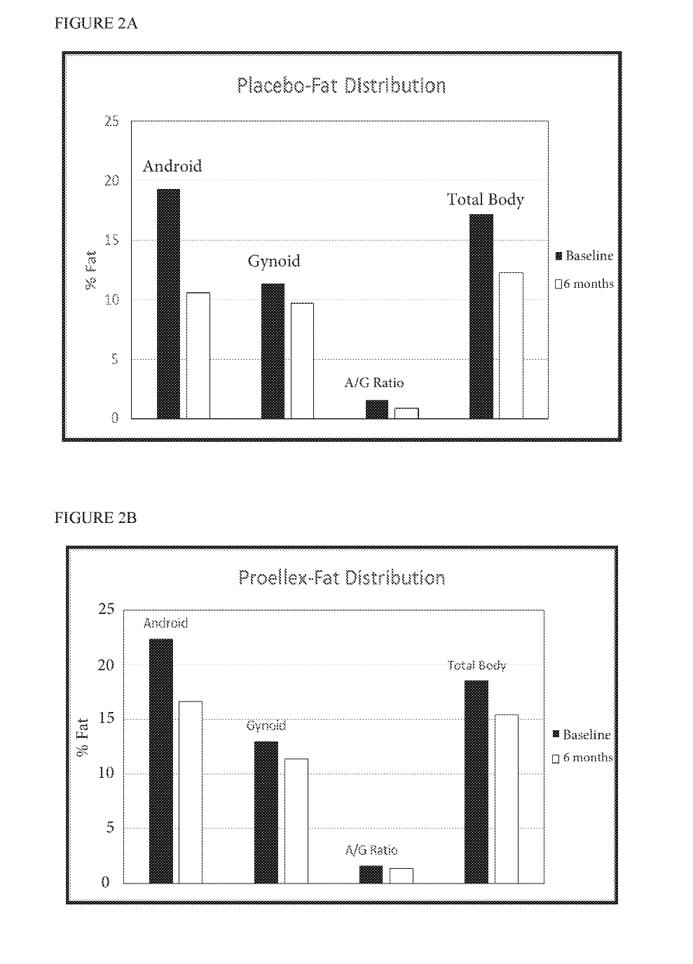

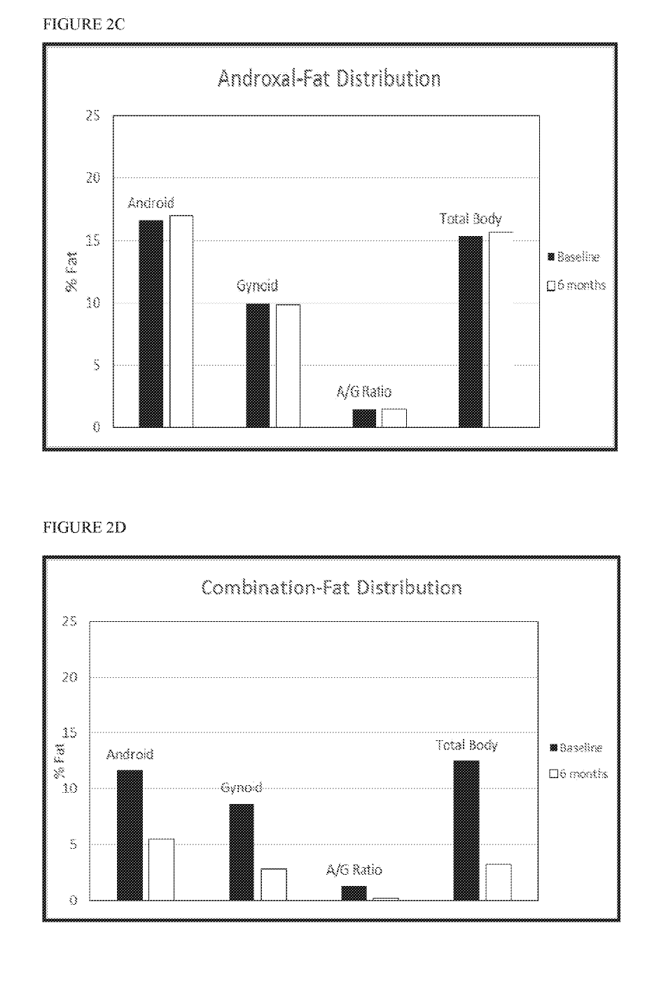

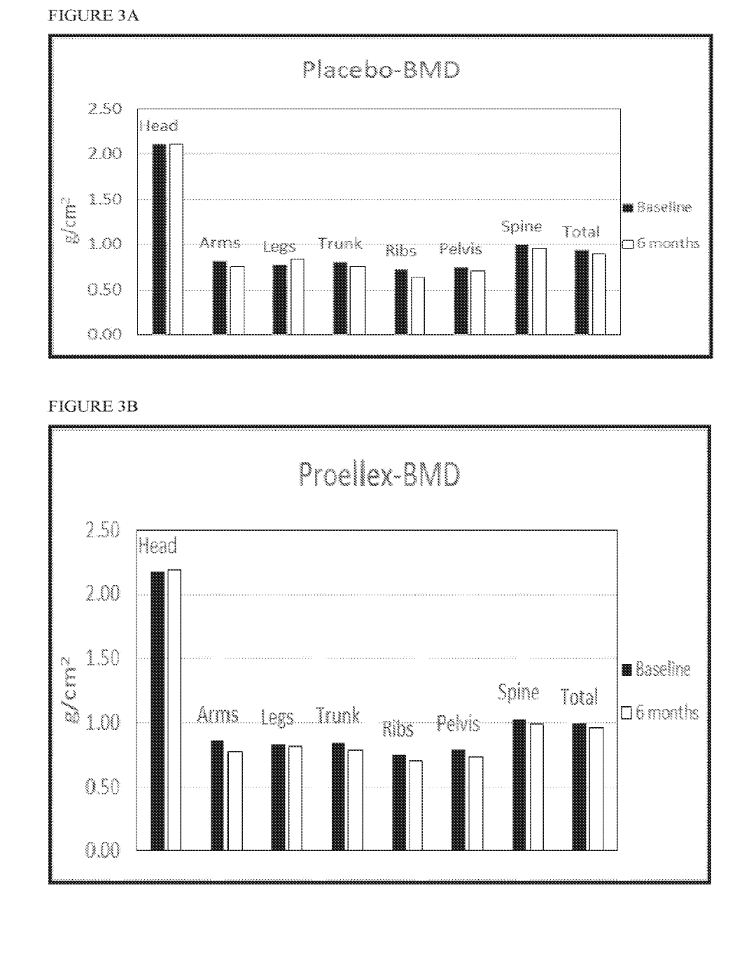

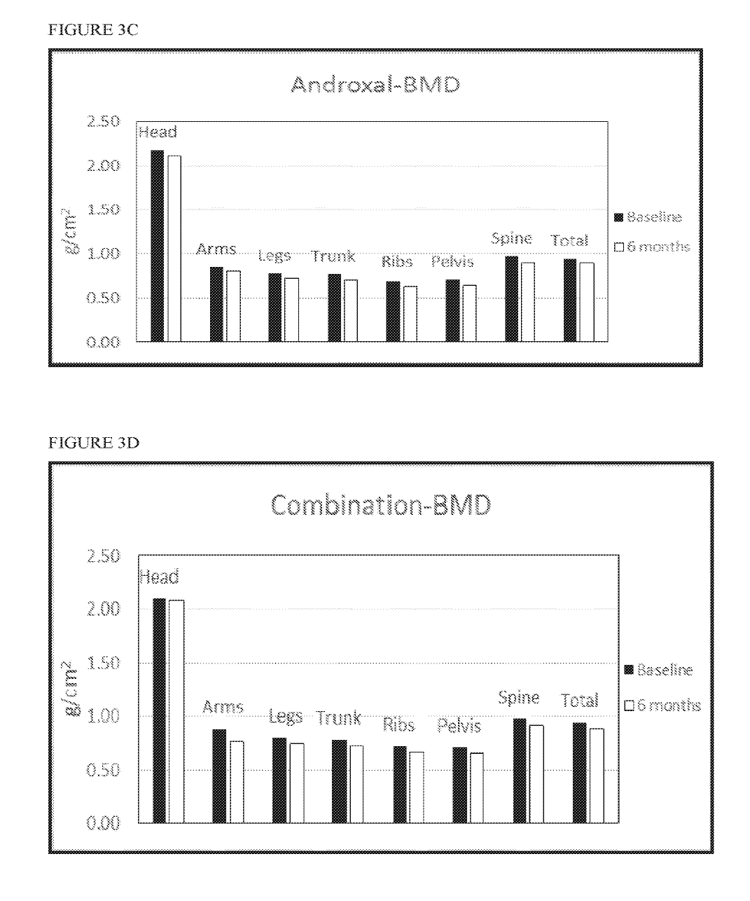

Assessment of the effects on body composition and bone mineral density by DEXA revealed no adverse effects on body composition or bone mineral density with the combination treatment. See FIGS. 1A-1D, 2A-2D and 3A-3D.

Treatment with Proellex alone resulted in an increase in endometrial thickness in the baboons similar to that previously observed in human females undergoing chronic treatment with Proellex and other SPRMs. The increase in endometrial thickness accompanying chronic treatment with Proellex was prevented to some degree with the combined treatment of Proellex and Androxal.

Importantly, enclomiphene prevented cycling in the baboons. It was observed that cycling of vaginal bleeding was stopped with administration of Proellex alone, enclomiphene alone and with the combination therapy. Bleeding resumed within a month of discontinuing the drugs.

It was also observed that enclomiphene and combination therapy but not proellex alone inhibited the swelling (turgesence, corresponding to the follicular phase of the ovarian cycle) of the tissue around the tail that is under the control of estrogen.

The data indicate that combination therapy with enclomiphene and an SPRM such as CDB-4124 prevents cycling in females and can be administered chronically without adverse effects on bone and endometrial tissue. The strong anti-hormonal pressure provided by the combination therapy provides a surprisingly effective and advantageous treatment for hormone dependent disorders such as endometriosis and pain associated therewith, adenomyosis, endometriosis or pain associated therewith, endometriomas of the ovary, dysmenorrhea, endocrine hormone-dependent tumors, uterine fibroids, endometrial hyperproliferation, menorrhagia, ovarian cancer, cervical cancer and breast cancer. A use for the combination in a method of contraception is also supported.

What is claimed is:

1. A method for treating a hormone-dependent condition in a mammal, comprising administering to a mammal in need thereof, an effective amount of a combination comprising (a) trans-clomiphene or a salt or analogue thereof and (b) a selective progesterone receptor modulator (SPRM), wherein the hormone-dependent condition is selected from the group consisting of: adenomyosis, endometriosis or pain associated therewith, endometriomas of the ovary, dysmenorrhea, endocrine hormone-dependent tumors, uterine fibroids, endometrial hyperproliferation, menorrhagia, ovarian cancer, cervical cancer and breast cancer.

2. A method of contraception comprising administering to a female mammal in need thereof, an effective amount of a combination comprising (a) trans-clomiphene or a salt or analogue thereof and (b) an SPRM.

3. The method of claim 1 or 2, wherein the SPRM is selected from telapristone (21 -methoxy-17α-acetoxy-11β-(4 N, N-dimethylaminophenyl)-19-norpregna-4,9-diene-3,20-dione), ulipristal (17α-acetoxy-11β-(4-N,N-dimethylaminophenyl)-19-norpregna-4,9-dien-3,20-dione), CDB-4453 (21-methoxy-17α-acetoxy-11β-(4-N-methylaminophenyl)-19-norpregna-4,9-diene-3,20-dione) and asoprisnil (benzaldehyde, 4-[(11β,17β)-17-methoxy-17-(methoxymethyl)-3-oxoestra-4,9-dien-11-yl]-1-(E)-oxim).

4. The method of claim 3, wherein the SPRM is telapristone.

5. The method of any one of claims 1-4, wherein (a) and (b) are sequentially administered.

6. The method according to any one of claims 1-4, wherein (a) and (b) are co-administered to the mammal in the same formulation.

7. The method according to any one of claims 1-6, wherein (a) and (b) are administered to the mammal by the same route, preferably wherein (a) and (b) are both administered orally.

8. The method according to any one of claims 1-7, wherein the combination comprises an analogue of trans-clomiphene.

9. The method according to claim 8, wherein the trans-clomiphene analogue is selected from: (E)-Clomiphene-NO, (E)-di-desethyl Clomiphene, (E)-desethyl Clomiphene, (E)-4-OH-Clomiphene and (E)-4-OH-desethyl Clomiphene.

10. The method according to claim 9, wherein the trans-clomiphene analogue is (E)-4-OH-Clomiphene or (E)-4-OH-desethyl Clomiphene.

11. The method according to any preceding claim wherein the SPRM is orally administered at a dose of from 3 mg to 30 mg, preferably, from 3 mg to 24 mg, from 3 mg to 12.5 mg, about 3 mg, about 6 mg, or about 12 mg per day or every other day.

12. The method according to any preceding claim wherein trans-clomiphene or a salt or analogue thereof is administered at a dose of 5 mg to 30 mg, 5 mg to 20 mg, 5 mg to 12 mg, 5 mg, 10 mg, 12 mg, 15 mg, 20 mg, 25 mg, or 30 mg per day or every other day.

13. A method according to any preceding claim, wherein the combination is administered continuously to the mammal for a period of at least 6 months, at least 7 months, at least 8 months, at least 9 months, at least one year, at least two years, at least three years, at least four years or at least five years.

14. The method according to any preceding claim, wherein the mammal is a human female.

15. A combination comprising (a) trans-clomiphene or a salt or analogue thereof and (b) a selective progesterone receptor modulator (SPRM), preferably telapristone, ulipristal, CDB-4453 or asoprisnil, for simultaneous, separate or sequential use.

16. A commercial package comprising the combination according to claim 15, together with instructions for simultaneous, separate or sequential use thereof.
